(19) United States
(12) Patent Application Publication
(10) Pub. No.: US 2023/0048204 A1
(43) Pub. Date: Feb. 16, 2023

(54) SKINCARE PRODUCT AND MULTI-DIMENSIONAL ASCORBIC ACID SERUM FORMULATION THEREFOR

(71) Applicant: Jiayin BAO, Newmarket (CA)

(72) Inventor: Jiayin BAO, Newmarket (CA)

(21) Appl. No.: 17/444,958

(22) Filed: Aug. 12, 2021

(51) Int. Cl. A61K 8/67; A61K 8/64; A61K 8/31; A61K 8/03; A61Q 19/00

(52) U.S. Cl. CPC A61K 8/676; A61K 8/03; A61K 8/31; A61K 8/64; A61K 8/678; A61Q 19/00

(57) ABSTRACT

A skincare product having packaged in a container a stable and effective ascorbic acid (vitamin C) serum formulation formulated from a skincare composition and a composition therefor. The composition comprises several types of hydrophilic ascorbyl (vitamin C) molecules in their respective suitably selected concentration ranges, several types of hydrophobic ascorbyl (vitamin C) molecules with their respective suitably selected concentration ranges, glutathione to co-stabilize the water phase vitamin C, bisabolol to prevent skin irritation from high vitamin C concentration, and a lipid cap that contains several types of vitamin E and squalane (non-oxidizable) to insulate hydrophilic/water phase from air.

### FIELD OF INVENTION

The invention relates generally to the field of skincare products and an improved composition and formulation therefor. In particular, the invention relates to an improved skincare composition, a stable and effective ascorbic acid (vitamin C) serum formulation formulated from the skincare composition and skincare products packaged with such a formulation.

### BACKGROUND OF INVENTION

Many skincare products are based on ascorbic acid (vitamin C) formulations or have ascorbic acid as an effective ingredient, for the reason that ascorbic acid is generally believed to have pronounced skin penetration power and a skin brightening effect. However, ascorbic acid is not stable due to oxidation. For example, it is known that pure ascorbic acid in the water type of solution is unstable, demonstrating color change and significant lost skin brightening activity within one to two weeks. Thus, if any skincare products are made from such water-based formulations, by the time they reach retail counters, which often takes more than two weeks, the products may already have turned yellow with minimal skin brightening activity.

There have been numerous efforts to improve stability of ascorbic acid based skincare products, but they are not ideal and sometimes introduce other undesirable effects. For example, to improve stability, there have been proposals to use single ingredient product with high concentration of ascorbic acid and in powder form. However, the powder form is still susceptible to oxidation. In addition, the powder formation is complex for concentration control and tends to cause skin irritation.

There have also been proposals to use vitamin E and ferulic acid to stabilize vitamin C in the formulation. However, ferulic acid can lead to skin irritation, too. In addition, a single type of vitamin C is not ideal for transversal diffusion into the skin, and there is still noticeable oxidation over time.

There are also non-aqueous formulations utilizing a hydrophobic solution to mix with single high concentration hydrophobic ascorbic acid analog to achieve more stable solution. However, despite improved stability, the hydrophobic ascorbyl analogs have inferior efficacy compared to the conventional ascorbic acid. In addition, the hydrophobic-based formulations can lead to greasy film on the skin of its user, which drastically reduces the attraction to consumers.

The forgoing creates challenges and constraints for providing a skincare product and formulation therefor that exhibit good stability and remain effective for long period of time, without introducing undesirable side effects. There is therefore a need for an improved skincare product, and formulation therefor, as compared to the existing art. It is an object of the present invention to mitigate or obviate at least one of the above mentioned disadvantages.

### SUMMARY OF INVENTION

The invention relates generally to a skincare product and an improved formulation therefor. In particular, the invention relates to multi-dimensional ascorbic acid (vitamin C) serum formulations and skincare products packaged with such a formulation.

It is desirable to create a stable and effective ascorbic acid (vitamin C) serum formulation for use in a skincare product that exhibit antioxidation and are effective to provide skin brightening effects after application. The product should not exhibit significant color change within certain minimum time period, such as within 12 months, which is a generally required shelf life for most consumer products. The efficacy should meet the requirement that visible skin brightening effect may be achieved after application. There also should not be undesirable side effects, such as skin irritation caused by high vitamin C concentration.

In one embodiment, as will be further described herein, a composition for skincare products, which may be formulated into a stable and effective ascorbic acid (vitamin C) serum formulation, comprises several types of hydrophilic ascorbyl (vitamin C) molecules, several types of hydrophobic ascorbyl (vitamin C) molecules, glutathione to co-stabilize the water phase vitamin C, bisabolol to prevent skin irritation from high vitamin C concentration, and a lipid cap that contains several types of vitamin E and squalane (non-oxidizable) to insulate hydrophilic/water phase of ascorbyl molecules from air.

In another embodiment, conventional ascorbic acid is combined with several different types of ascorbyl analogs so that various ascorbyl analogs can stabilize the ascorbic acid to maintain efficacy. In addition to ascorbyl analogs, other antioxidant small molecules such as squalane, vitamin E, glutathione may be blended into one of such formulations to stabilize and enhance the efficacy of ascorbyl species at the same time. As used herein, a “small molecule” is a low molecular weight (less than about 1000 daltons) organic compound that may regulate a biological process, with a size on the order of 1 nm. A chemical analog is a compound having a structure like that of another compound but differing from it in respect to a certain component. For example, the L-ascorbic acid and magnesium ascorbyl phosphate are chemical analogs to each other.

In a cosmetic or skincare product including a container and an ascorbic acid (vitamin C) serum formulation packaged therein, the serum formulation takes a bi-layer form, having a hydrophilic/water phase that is substantially water based and a hydrophobic/lipid phase that is substantially oil based, the hydrophilic/water phase includes several types of hydrophilic ascorbyl (vitamin C) molecules and additional hydrophilic antioxidant or antioxidants to co-stabilize the water phase vitamin C, the hydrophobic/lipid phase includes several types of hydrophobic ascorbyl (vitamin C) molecules and antioxidants such as different types of vitamin E and squalane (non-oxidizable). In the normal upright storage position, the hydrophilic/water phase is located at the bottom and the hydrophilic/water phase is located on top of the hydrophilic/water phase, while the lipid phase antioxidants form a lipid cap to insulate hydrophilic/water phase of ascorbyl molecules from air. The lipid cap thus physically seals the aqueous ascorbyl molecules located at the bottom and acts to prevent air molecules from entering the hydrophilic/water phase to enhance stability and thus maintain longer period of efficacy. The serum formulation may further include a skin soothing agent such as bisabolol to reduce and prevent skin irritation from high vitamin C concentration.

As used herein, the term “substantially” and its variations mean largely but not necessarily wholly as understood by one of ordinary skill in the art, and in a non-limiting embodiment, “substantially” refers to concentration ranges of more than 90%, more than 95%, more than 99%, more than 99.5%, more than 99.9%, or even higher concentrations. The term “concentration” as used herein means a percentage by weight, of the composition. For example, when stating that a substance (or a category of substances) has a concentration of 95%, it means that the substance (or the category of the substances) is present in the composition in an amount of 95% by weight relative to the total weight of the composition, or by weight of the composition, in short. Unless otherwise noted, all percentages used herein are by weight of the composition and all measurements refer to that made at room temperature, namely between 20° C. and 25° C. The term “about” modifying the amount or percentage of an ingredient in a composition refers to variation in the numerical quantity or accuracy that can occur, for example, through typical measuring and handling procedures used for making or using compositions in the real world, or through ordinary rounding approaches and in light of the number of significant digits, and the like. Other than in the examples not expressed using a range, or where otherwise indicated, all numbers expressing percentages or quantities of ingredients and so forth used in the specification and claims are to be understood as being modified in all instances by the term “about.”

As would be understood by one of ordinary skill in the art, many variations of nomenclature are commonly used to refer to a specific chemical composition. Accordingly, commonly used alternative names may be provided in quotations and parentheses/brackets, adjacent to a chemical composition’s preferred name when referred to herein. Unless otherwise noted, all technical and scientific terms used herein have the same meanings as commonly understood by one of ordinary skill in the art. As used herein, the terms “mixture”, “mix”, and “mixing” or any variants thereof include stirring, blending, dispersing, milling, homogenizing, and other similar methods. Ingredients may be categorized or described herein by their therapeutic benefit or their chemical functions. However, it is to be understood that any ingredients useful herein may provide more than one benefit or operate via more than one mode of action. Therefore, classifications or categorizations herein are made for the sake of convenience and are not intended to limit an ingredient to any particularly stated application or applications listed.

In a first aspect of the invention, a composition for a skincare product is provided. The composition contains one or more hydrophilic ascorbyl (vitamin C) molecules, one or more hydrophobic ascorbyl (vitamin C) molecules, one or more hydrophilic antioxidants to co-stabilize the one or more hydrophilic ascorbyl molecules, and a lipid cap comprising one or more hydrophobic antioxidants. The hydrophilic ascorbyl (vitamin C) molecules may be selected from L-ascorbic acid, magnesium ascorbyl phosphate, sodium ascorbyl phosphate, calcium L-ascorbate dihydrate, sodium L-ascorbate, ascorbyl glucoside, glyceryl ascorbate, and 3-O-ethyl-L-ascorbic acid and the hydrophobic ascorbyl (vitamin C) molecules may be selected from L-ascorbyl palmitate, ascorbyl tetraisopalmitate, and tetrahexyldecyl ascorbate. The hydrophilic ascorbyl molecules and the hydrophobic ascorbyl (vitamin C) molecules may have a total combined concentration of from 10% to 40% by weight of the composition, or about 15% by weight. The antioxidants may be selected from or comprise glutathione, squalane, tocopherol, and tocopheryl acetate. A skin soothing agent also may be added.

In a second aspect, a composition for a skincare product comprises hydrophilic ascorbyl (vitamin C) molecules comprising L-ascorbic acid, magnesium ascorbyl phosphate, sodium ascorbyl phosphate, calcium L-ascorbate dihydrate, sodium L-ascorbate, ascorbyl glucoside, glyceryl ascorbate, and 3-O-ethyl-L-ascorbic acid, hydrophobic ascorbyl (vitamin C) molecules comprising L-ascorbyl palmitate, ascorbyl tetraisopalmitate, and tetrahexyldecyl ascorbate, a hydrophilic antioxidant comprising glutathione, and lipid phase antioxidants comprising squalane, tocopherol, and tocopheryl acetate, wherein the lipid phase antioxidants forming a lipid cap. According to one feature of this aspect of the invention, the L-ascorbic acid is present in an amount from 1% to 30% by weight of the composition, each one of magnesium ascorbyl phosphate, sodium ascorbyl phosphate, calcium L-ascorbate dihydrate, sodium L-ascorbate, ascorbyl glucoside, glyceryl ascorbate, and 3-O-ethyl-L-ascorbic acid is present in an amount from 0.1% to 3% by weight of the composition, the glutathione is present in an amount from 1% to 10% by weight of the composition, the squalane is present in an amount from 1% to 30% by weight of the composition, and each one of the tocopherol, the tocopheryl acetate, the hydrophobic ascorbyl molecules is present in an amount from 0.1% to 3% by weight of the composition. The hydrophilic ascorbyl molecules and the hydrophobic ascorbyl molecules may have a total combined concentration of 15% by weight of the composition.

In a third aspect of the invention, there is provided a skincare product comprising a container and a bi-layer serum formulation stored therein. The bi-layer serum formulation is formed from a composition that has a hydrophilic/water phase and a hydrophobic/lipid phase. The hydrophilic/water phase includes a hydrophilic ascorbyl mixture of hydrophilic ascorbyl molecules which may be selected from L-ascorbic acid, magnesium ascorbyl phosphate, sodium ascorbyl phosphate, calcium L-ascorbate dihydrate, sodium L-ascorbate, ascorbyl glucoside, glyceryl ascorbate, and 3-O-ethyl-L-ascorbic acid; the hydrophobic/lipid phase includes a hydrophobic lipid mixture of hydrophilic ascorbyl (vitamin C) molecules selected from L-ascorbyl palmitate, ascorbyl tetraisopalmitate, and tetrahexyldecyl ascorbate. The formulation further includes one or more antioxidant small molecules selected from squalane, tocopherol, and tocopheryl acetate, which forms a lipid cap to seal the hydrophilic/water phase from air. Skin soothing agents, such as bisabolol, may be added to reduce skin irritation caused by ascorbyl molecules.

In other aspects the invention provides various combinations and subsets of the aspects described above.

### BRIEF DESCRIPTION OF DRAWINGS

For the purposes of description, but not of limitation, the foregoing and other aspects of the invention are explained in greater detail with reference to the accompanying drawings, in which:

Fig. 1 is a schematic diagram showing a skincare product package described herein.

### DETAILED DESCRIPTION OF EMBODIMENTS

The description which follows and the embodiments described therein are provided by way of illustration of an example, or examples, of particular embodiments of the principles of the present invention. These examples are provided for the purposes of explanation, and not limitation, of those principles and of the invention.

In general, it is desirable that a stable and effective ascorbic acid (vitamin C) serum formulation should meet the requirements for stability, efficacy and minimum skin irritation. In general, the product should not exhibit any significant color change within a minimum time period, such as within 12 months, a typical period that is the generally required shelf life for most consumer products. The efficacy should meet the requirement that visible skin brightening effect may be achieved after application. There also should not be undesirable side effects such as skin irritation due to high vitamin C concentration or formation of greasy film on the skin of its user.

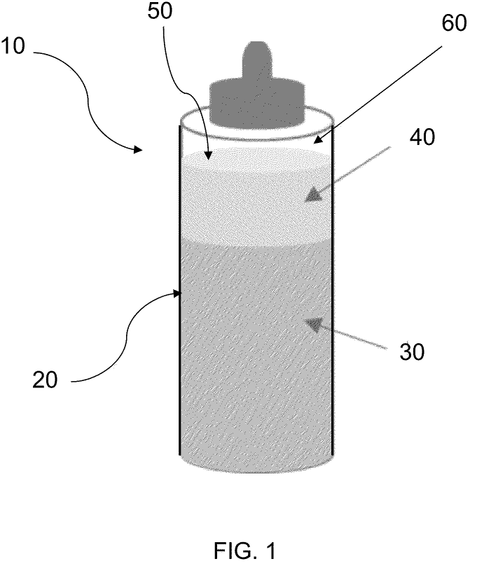

Fig. 1 shows a cosmetic or skincare product 10 packaged in a container 20. A skincare composition, formulated as a stable and effective ascorbic acid (vitamin C) serum formulation, is packaged in the container. The formulation has a bi-layer or heterogeneous liquid phase form. The bi-layer serum formulation has a hydrophilic/water phase and a hydrophobic/lipid phase.

The hydrophilic/water phase is located at the bottom and forms a bottom layer 30. The bottom layer is substantially water based. This bottom layer 30 may comprise as much as 99% (or even higher concentration) hydrophilic ascorbyl mixture, though in general, it may be about 90% hydrophilic ascorbyl mixture, or even as low as 80% hydrophilic ascorbyl mixture. The hydrophilic ascorbyl mixture may include one or more of hydrophilic ascorbyl molecules, such as L-ascorbic acid, magnesium ascorbyl phosphate, sodium ascorbyl phosphate, calcium L-ascorbate dihydrate, sodium L-ascorbate, ascorbyl glucoside, glyceryl ascorbate, and 3-O-ethyl-L-ascorbic acid.

The hydrophobic/lipid phase is generally located on top of the water phase in normal upright storage conditions and forms a top layer 40. The top layer 40 is substantially oil based. The top layer 40 may comprise as much as 99% (or even higher concentration) hydrophobic lipid mixture, though in general, it may be about 90% hydrophobic lipid mixture, or even as low as 80% hydrophobic lipid mixture. The hydrophobic lipid mixture may include one or more hydrophobic ascorbyl (vitamin C) molecules such as L-ascorbyl palmitate, ascorbyl tetraisopalmitate, and tetrahexyldecyl ascorbate. The hydrophobic lipid mixture also may include hydrophobic antioxidants. The hydrophobic antioxidants are non-oxidizable which can be used to seal physically the aqueous ascorbyl mixture in the bottom layer. Accordingly, the hydrophobic small molecule moieties form an antioxidation air isolation cap 50 that can prevent air 60 from entering the ascorbyl mixture 30. This lipid cap physically seals the aqueous ascorbyl mixture 30 located at the bottom, thus enhancing its stability and maintaining longer period of efficacy of the skincare product.

As will be understood by those skilled in the art, although references are made to hydrophilic/water phase and hydrophobic/lipid phase, these phases tend to form and become separated when the skincare product 10 is positioned in the normal upright storage conditions. The formulation itself may be made simply by directly mixing the selected hydrophilic ascorbyl molecules, the selected hydrophobic ascorbyl molecules, and the antioxidants, the skin soothing agent (such as bisabolol), without having to form first a hydrophilic ascorbyl mixture (or phase) nor a hydrophobic lipid mixture (or phase), though such mixtures may be first formed for convenience.

A composition for the skincare product 10 thus may comprise one or more types of hydrophilic ascorbyl (vitamin C) molecules, one or more types of hydrophobic ascorbyl (vitamin C) molecules, one or more hydrophilic antioxidants such as glutathione to co-stabilize the water phase vitamin C, and a lipid cap that contains one or more hydrophobic antioxidants, such as different types of vitamin E and squalane (non-oxidizable), which helps to insulate hydrophilic/water phase from air. The composition may further include a skin soothing agent such as bisabolol to prevent skin irritation from high vitamin C concentration.

Hydrophilic ascorbyl mixture, which forms part of the bottom layer or hydrophilic/water phase, may be selected from different hydrophilic ascorbyl (vitamin C) molecules, such as L-ascorbic acid, magnesium ascorbyl phosphate, sodium ascorbyl phosphate, calcium L-ascorbate dihydrate, sodium L-ascorbate, ascorbyl glucoside, glyceryl ascorbate, 3-O-ethyl-L-ascorbic acid, among others, with suitable selected concentrations. In general, L-ascorbic acid may have a concentration in the range of 1% to 30% by weight, while other hydrophilic ascorbyl molecules may each have a concentration in the range of 0.1% to 3% by weight.

Hydrophobic lipid mixture, which forms part of the top layer or hydrophobic/lipid phase, may be selected from different hydrophobic ascorbyl (vitamin C) molecules, such as L-ascorbyl palmitate, ascorbyl tetraisopalmitate, tetrahexyldecyl ascorbate. Additionally, lipid phase antioxidants, such as squalane, tocopherol, tocopheryl acetate, among others, may be selected, which form a lipid cap. Squalane may have a concentration in the range of 1% to 30% by weight, while other hydrophobic ascorbyl molecules and lipid phase antioxidants may each have a concentration in the range of 0.1% to 3% by weight.

In this formulation, as described above, the L-ascorbic acid is the main contributor in the formulation as it is believed to have high efficacy and bio availability. In one embodiment, the hydrophilic ascorbyl (vitamin C) molecules and the hydrophobic ascorbyl (vitamin C) molecules have a total combined concentration of 15% by weight, i.e., a 15% overall vitamin C concentration in the formulation, though the total combined vitamin C concentration may be in the range of 10% to 20%, or 10% to 40%, or even 5% to 40%, too, as suitable.

Antioxidants help to achieve synergistic co-stabilization. Added to the formulation are preferably antioxidant small molecules. These antioxidant small molecules are mixed or blended into the formulation to stabilize and enhance the efficacy of ascorbyl species at the same time. In general, a small molecule is a low molecular weight (< 1000 daltons) organic compound that may regulate a biological process, with a size on the order of 1 nm. Suitable antioxidant small molecules may include glutathione, which is a water phase antioxidant, and squalane, vitamin E, tocopherol, tocopheryl acetate, among others, which are lipid phase antioxidants. Glutathione may have a concentration in the range of 1% to 10% by weight, squalane may have a concentration in the range of 1% to 30% by weight, and each of the other lipid phase antioxidants may have a concentration in the range of 0.1% to 3% by weight.

In one embodiment, both water phase antioxidants and lipid phase antioxidants are included in the selected antioxidant small molecules. However, it is also acceptable to select antioxidant small molecules without including water phase antioxidants, such as without including glutathione.

To reduce skin irritation, generally due to high vitamin C concentration, skin soothing agents, such as bisabolol, may be added. Skin soothing agents may be a hydrophobic type and form part of the hydrophobic/lipid phase. The addition of bisabolol in the formulation can soothe irritation that may be caused by acidity due to high vitamin C concentration. The bisabolol may have a concentration in the range of 0.1% to 3% by weight.

Table 1 below illustrates as an example a list of ingredients and their respective concentration ranges in a composition which can be formulated for the skincare product 10:

Table 1

INGREDIENTS
HYDROPHILIC/WATER PHASE

| L-ASCORBIC ACID | 1-30% | ASCORBYL MOLECULES |
|---|---|---|
| MAGNESIUM ASCORBYL PHOSPHATE | 0.1-3% | |
| SODIUM ASCORBYL PHOSPHATE | 0.1-3% | |
| CALCIUM L-ASCORBATE DIHYDRATE | 0.1-3% | |
| SODIUM L-ASCORBATE | 0.1-3% | |
| ASCORBYL GLUCOSIDE | 0.1-3% | |
| GLYCERYL ASCORBATE | 0.1-3% | |
| 3-O-ETHYL-L-ASCORBIC ACID | 0.1-3% | |
| GLUTATHIONE | 1-10% | ANTIOXIDANT |
| HYDROPHOBIC/LIPID PHASE | | |
| SQUALANE | 1-30% | ANTIOXIDANT |
| TOCOPHEROL | 0.1-3% | |
| TOCOPHERYL ACETATE | 0.1-3% | |
| L-ASCORBYL PALMITATE | 0.1-3% | ASCORBYL MOLECULES |
| ASCORBYL TETRAISOPALMITATE | 0.1-3% | |
| TETRAHEXYLDECYL ASCORBATE | 0.1-3% | |
| BISABOLOL | 0.1-3% | REDUCE IRRITATION |

Table 1 lists some examples of hydrophilic ascorbyl molecules and hydrophobic ascorbyl molecules. Of course, it will be understood that other ascorbyl molecules or ascorbyl analogs also may be selected for the formulation. Further, although the table listed eleven (11) different types of ascorbyl molecules, it will be understood that not all eleven of them must be selected for the formulation. For example, in one embodiment, a formulation is formed from six (6) different types of ascorbyl analogs in combination with a conventional vitamin C (L-ascorbic acid). Also included in this embodiment are four different antioxidants, of which squalane, tocopherol, and tocopheryl acetate together form a lipid cap. Table 2 below shows the ingredients and their respective concentrations in such a formulation:

Table 2

| INGREDIENTS | Concentration |
|---|---|
| L-ASCORBIC ACID | 9 |
| MAGNESIUM ASCORBYL PHOSPHATE | 2 |
| SODIUM ASCORBYL PHOSPHATE | 0.1 |
| CALCIUM L-ASCORBATE DIHYDRATE | 0.1 |
| SODIUM L-ASCORBATE | 0.1 |
| ASCORBYL GLUCOSIDE | 0.1 |
| GLYCERYL ASCORBATE | 0.1 |
| 3-O-ETHYL-L-ASCORBIC ACID | 0.1 |
| GLUTATHIONE | 1 |
| SQUALANE | 9 |
| TOCOPHEROL | 0.1 |
| TOCOPHERYL ACETATE | 0.1 |

When a conventional ascorbic acid is combined with multiple types of ascorbyl analogs, presence of these different types of ascorbyl analogs helps to stabilize the ascorbic acid. It is considered possible that the addition of ascorbyl analogs can replenish the ascorbic acid by converting ascorbyl analogs into the ascorbic acid when the latter is depleted from oxidation. Thus, various ascorbyl analogs tend to stabilize the ascorbic acid to maintain the formulation’s efficacy. Similarly, multiple types of vitamin C, when mixed together in a combination, also tend to co-stabilize each other. In general, it is preferred that at least two hydrophilic ascorbyl molecules are selected.

Table 3 shows the ingredients and their corresponding concentrations of a formulation according to another embodiment.

Table 3

| INGREDIENTS | CONCENTRATION |
|---|---|
| HYDROPHILIC PHASE | |
| AQUA | 72.95 |
| L-ASCORBIC ACID | 9 |
| MAGNESIUM ASCORBYL PHOSPHATE | 2 |
| SODIUM ASCORBYL PHOSPHATE | 0.1 |
| CALCIUM L-ASCORBATE DIHYDRATE | 0.1 |
| SODIUM L-ASCORBATE | 0.1 |
| ASCORBYL GLUCOSIDE | 0.1 |
| GLYCERYL ASCORBATE | 0.1 |
| 3-O-ETHYL-L-ASCORBIC ACID | 0.1 |
| TRANEXAMIC ACID | 1 |
| SODIUM PCA | 0.5 |
| SODIUM POLYGLUTAMATE | 0.5 |
| PANTHENOL | 0.5 |
| CERAMIDE NP | 0.5 |
| GLUTATHIONE | 1 |
| PHENOXYETHANOL SA | 1.5 |
| HYDROPHOBIC PHASE | |
| SQUALANE | 9 |
| TOCOPHEROL | 0.1 |
| TOCOPHERYL ACETATE | 0.1 |
| L-ASCORBYL PALMITATE | 0.05 |
| ASCORBYL TETRAISOPALMITATE | 0.1 |
| TETRAHEXYLDECYL ASCORBATE | 0.1 |
| BISABOLOL | 0.5 |

100

Again, it will be understood by those skilled in the art that the listing of ingredients in Table 3, their inclusion in the listing, and their respective concentrations, are merely used to illustrate the invention and that the invention is not to be limited by this or any of the other examples.

Various embodiments of the invention have now been described in detail. Those skilled in the art will appreciate that numerous modifications, adaptations and variations may be made to the embodiments without departing from the scope of the invention, which is defined by the appended claims. The scope of the claims should be given the broadest interpretation consistent with the description as a whole and not to be limited to these embodiments set forth in the examples or detailed description thereof.

What is claimed is:

1. A composition for a skincare product, comprising:
one or more hydrophilic ascorbyl (vitamin C) molecules,
one or more hydrophobic ascorbyl (vitamin C) molecules,
one or more hydrophilic antioxidants to co-stabilize the one or more hydrophilic ascorbyl molecules, and
a lipid cap comprising one or more hydrophobic antioxidants.

2. The composition of claim 1, wherein
the one or more hydrophilic ascorbyl (vitamin C) molecules are selected from the group consisting of: L-ascorbic acid, magnesium ascorbyl phosphate, sodium ascorbyl phosphate, calcium L-ascorbate dihydrate, sodium L-ascorbate, ascorbyl glucoside, glyceryl ascorbate, and 3-O-ethyl-L-ascorbic acid.

3. The composition of claim 1, wherein the one or more hydrophilic ascorbyl (vitamin C) molecules comprise L-ascorbic acid, magnesium ascorbyl phosphate, sodium ascorbyl phosphate, calcium L-ascorbate dihydrate, sodium L-ascorbate, ascorbyl glucoside, glyceryl ascorbate, and 3-O-ethyl-L-ascorbic acid.

4. The composition of claim 3, wherein the L-ascorbic acid is present in an amount from 1% to 30% by weight of said composition, and each one of magnesium ascorbyl phosphate, sodium ascorbyl phosphate, calcium L-ascorbate dihydrate, sodium L-ascorbate, ascorbyl glucoside, glyceryl ascorbate, and 3-O-ethyl-L-ascorbic acid is present in an amount from 0.1% to 3% by weight of said composition.

5. The composition of claim 4, wherein the one or more hydrophilic ascorbyl molecules and the one or more hydrophobic ascorbyl (vitamin C) molecules have a total combined concentration of from 10% to 40% by weight of said composition.

6. The composition of claim 5, wherein the total combined concentration is 15%.

7. The composition of claim 1, wherein
the one or more hydrophobic ascorbyl (vitamin C) molecules are selected from the group consisting of: L-ascorbyl palmitate, ascorbyl tetraisopalmitate, and tetrahexyldecyl ascorbate.

8. The composition of claim 1, wherein the one or more hydrophobic ascorbyl (vitamin C) molecules comprise L-ascorbyl palmitate, ascorbyl tetraisopalmitate, and tetrahexyldecyl ascorbate.

9. The composition of claim 8, wherein each one of L-ascorbyl palmitate, ascorbyl tetraisopalmitate, and tetrahexyldecyl ascorbate is present in an amount from 0.1% to 3% by weight of said composition.

10. The composition of claim 9, wherein the one or more hydrophilic ascorbyl molecules and the one or more hydrophobic ascorbyl (vitamin C) molecules have a total combined concentration of from 10% to 40% by weight of said composition.

11. The composition of claim 10, wherein the total combined concentration is 15%.

12. The composition of claim 1, wherein glutathione is selected as the one or more hydrophilic antioxidants and the one or more lipid phase antioxidants is selected from the group consisting of squalane, tocopherol, and tocopheryl acetate.

13. The composition of claim 1, wherein glutathione is selected as the one or more hydrophilic antioxidants and the one or more lipid phase antioxidants comprises squalane, tocopherol, and tocopheryl acetate.

14. The composition of claim 13, wherein the glutathione is present in an amount from 1% to 10% by weight of said composition, the squalane is present in an amount from 1% to 30% by weight of said composition, and the one or more hydrophilic ascorbyl (vitamin C) molecules and the one or more hydrophobic ascorbyl (vitamin C) molecules have a total combined concentration of from 10% to 40% by weight of said composition.

15. The composition of claim 14, wherein the total combined concentration is 15% and the composition further comprises a skin soothing agent.

16. A composition for a skincare product, comprising:
hydrophilic ascorbyl (vitamin C) molecules comprising L-ascorbic acid, magnesium ascorbyl phosphate, sodium ascorbyl phosphate, calcium L-ascorbate dihydrate, sodium L-ascorbate, ascorbyl glucoside, glyceryl ascorbate, and 3-O-ethyl-L-ascorbic acid,
hydrophobic ascorbyl (vitamin C) molecules comprising L-ascorbyl palmitate, ascorbyl tetraisopalmitate, and tetrahexyldecyl ascorbate,
a hydrophilic antioxidant comprising glutathione, and
lipid phase antioxidants comprising squalane, tocopherol, and tocopheryl acetate.

17. The composition of claim 16, wherein the hydrophilic ascorbyl molecules and the hydrophobic ascorbyl molecules have a total combined concentration of from 10% to 40% by weight of said composition.

18. The composition of claim 16, further comprising bisabolol in an amount from 0.1% to 3% by weight of said composition, wherein the L-ascorbic acid is present in an amount from 1% to 30% by weight of said composition, each one of magnesium ascorbyl phosphate, sodium ascorbyl phosphate, calcium L-ascorbate dihydrate, sodium L-ascorbate, ascorbyl glucoside, glyceryl ascorbate, and 3-O-ethyl-L-ascorbic acid is present in an amount from 0.1% to 3% by weight of said composition, the glutathione is present in an amount from 1% to 10% by weight of said composition, the squalane is present in an amount from 1% to 30% by weight of said composition, and each one of the tocopherol, the tocopheryl acetate and the hydrophobic ascorbyl molecules is present in an amount from 0.1% to 3% by weight of said composition.

19. The composition of claim 18, wherein the hydrophilic ascorbyl molecules and the hydrophobic ascorbyl molecules have a total combined concentration of 15% by weight of said composition.

20. A skincare product comprising a container and a bi-layer serum formulation stored therein, the bi-layer serum formulation being formed from a composition and having a hydrophilic/water phase and a hydrophobic/lipid phase,
the hydrophilic/water phase comprising a hydrophilic ascorbyl mixture of hydrophilic ascorbyl molecules selected from the group consisting of: L-ascorbic acid, magnesium ascorbyl phosphate, sodium ascorbyl phosphate, calcium L-ascorbate dihydrate, sodium L-ascorbate, ascorbyl glucoside, glyceryl ascorbate, and 3-O-ethyl-L-ascorbic acid,
the hydrophobic/lipid phase comprising a hydrophobic lipid mixture of hydrophilic ascorbyl (vitamin C) molecules selected from the group consisting of: L-ascorbyl palmitate, ascorbyl tetraisopalmitate, and tetrahexyldecyl ascorbate,
the formulation further comprising one or more antioxidant small molecules selected from the group consisting of: squalane, tocopherol, and tocopheryl acetate,
wherein the one or more selected antioxidant small molecules forms a lipid cap to seal the hydrophilic/water phase from air.

21. The skincare product of claim 20, wherein
the hydrophilic/water phase comprises L-ascorbic acid, magnesium ascorbyl phosphate, sodium ascorbyl phosphate, calcium L-ascorbate dihydrate, sodium L-ascorbate, ascorbyl glucoside, glyceryl ascorbate, and 3-O-ethyl-L-ascorbic acid,
the hydrophobic/lipid phase comprises L-ascorbyl palmitate, ascorbyl tetraisopalmitate, and tetrahexyldecyl ascorbate,
the one or more antioxidant small molecules comprise glutathione, squalane, tocopherol, and tocopheryl acetate, and
the formulation further comprises biasbolol,
wherein the L-ascorbic acid is present in an amount from 1% to 30% by weight of said composition, each one of magnesium ascorbyl phosphate, sodium ascorbyl phosphate, calcium L-ascorbate dihydrate, sodium L-ascorbate, ascorbyl glucoside, glyceryl ascorbate, and 3-O-ethyl-L-ascorbic acid is present in an amount from 0.1% to 3% by weight of said composition, the glutathione is present in an amount from 1% to 10% by weight of said composition, the squalane is present in an amount from 1% to 30% by weight of said composition, and each one of the tocopherol, the tocopheryl acetate, the bisabolol and the hydrophobic ascorbyl molecules is present in an amount from 0.1% to 3% by weight of said composition.

22. The skincare product of claim 21, wherein
the L-ascorbic acid is present in an amount of 9% by weight,
the magnesium ascorbyl phosphate is present in an amount of 2% by weight,
the sodium ascorbyl phosphate is present in an amount of 0.1% by weight,
the calcium L-ascorbate dihydrate is present in an amount of 0.1% by weight,
the sodium L-ascorbate is present in an amount of 0.1% by weight,
the ascorbyl glucoside is present in an amount of 0.1% by weight,
the glyceryl ascorbate is present in an amount of 0.1% by weight,
the 3-O-ethyl-L-ascorbic acid is present in an amount of 0.1% by weight,
the glutathione is present in an amount of 1% by weight,
the squalane is present in an amount of 9% by weight,
the tocopherol is present in an amount of 0.1% by weight,
the tocopheryl acetate is present in an amount of 0.1% by weight,
the L-ascorbyl palmitate is present in an amount of 0.05% by weight,
the ascorbyl tetraisopalmitate is present in an amount of 0.1% by weight,
the tetrahexyldecyl ascorbate is present in an amount of 0.1% by weight,
the bisabolol is present in an amount of 0.5% by weight, and
wherein the composition further comprises:
tranexamic acid in an amount of 1% by weight,
sodium pca in an amount of 0.5% by weight,
sodium polyglutamate in an amount of 0.5% by weight,
panthenol in an amount of 0.5% by weight, and
ceramide np in an amount of 0.5% by weight.
